(19) United States
(12) Patent Application Publication
(10) Pub. No.: US 2024/0036591 A1
(43) Pub. Date: Feb. 1, 2024

(54) SYSTEMS AND METHODS FOR VARIABLE FLOW RESISTANCE FOR A PUMP

(71) Applicant: PAVmed, Inc., New York, NY (US)

(72) Inventors: Lishan Aklog, New York, NY (US); Julian Fricks, New York, NY (US); Peter Aliski, New York, NY (US); Richard Yazbeck, New York, NY (US); Jessie Gifford, New York, NY (US)

(21) Appl. No.: 18/275,889

(22) PCT Filed: Feb. 4, 2022
(86) PCT No.: PCT/US22/15257

§ 371 Date: Aug. 4, 2023

Related U.S. Application Data
(60) Provisional application No. 63/145,814, filed on Feb. 4, 2021.

(51) Int. Cl. G05D 7/01; A61M 5/148

(52) U.S. Cl. CPC G05D 7/014; A61M 5/148

(57) ABSTRACT

Systems and methods for managing flow along a pathway are provided herein. The system and method can include a pump for providing a fluid flow along the pathway, a flow regulator in fluid communication at its input end with the pump and designed to maintain flow therethrough at a substantially constant flow rate, and the pathway being in fluid communication with an output of the flow resistor for directing fluid flow to a destination at the substantially constant flow rate.

### CROSS-REFERENCE TO RELATED APPLICATION(S)

This application claims priority to, and the benefit of, U.S. Provisional Application No. 63/145,814, filed Feb. 4, 2021, for all subject matter common to both applications. The disclosure of said provisional application is hereby incorporated by reference in its entirety.

### FIELD OF THE INVENTION

The present disclosure relates to systems and methods suitable for managing flow along a pathway. In particular, the present disclosure relates to systems and methods for adjusting flow provided by a pump.

### BACKGROUND

Many of fluid transfer applications require that a fluid flow is controlled to deliver a substance to a location at a specified rate. Flow can be controlled by setting the pressure differential, the resistance, or both. These can be actively controlled, but such systems require active pressure sources (e.g., pumps) or resistors (e.g., valves) often with feedback loops based on flow sensors.

Controlling flow completely passively, however, is more difficult. Passive flow resistors (e.g., manual or fixed valves, orifice plates, etc.) are commonly used to control flow but their accuracy are dependent on maintaining a fairly constant pressure. This is typically accomplished with a large reservoir of fluid, (relative to the volume of fluid to be delivered) with stored potential energy that is constant (e.g., elevated tank). A major limitation of this passive variable resistor design is that it is structurally linked to the infusion device and its design is dependent on the device. Perhaps more importantly, its specifications are dependent on the initial conditions, specifically the initial pressure, and the specific trajectory of the pressure for that specific device. The functionality of passive variable resistors would be greatly enhanced and available to a broader set of applications if its design and structure were independent of the pressure source and fluid reservoir and that its resistance was simply a function of the instantaneous pressure difference P at least over a specified range.

One example of a fluid transfer application is patient infusions. Infusions remain ubiquitous in healthcare spanning a wide range of conditions, substances, access sites and venues. Despite advances in oral and other drug delivery modes (e.g., transdermal, inhaled) many critical therapies still require intravenous (IV) infusion. It is estimated that one million infusions are administered per day in the United States. Over 90% of hospitalized patients receive an IV infusion. Infused substances can include drugs (e.g., antibiotics, chemotherapy, pain medications, local anesthetics, vasoactive agents, biologics), fluids (e.g., crystalloids, colloids, parenteral nutrition), and blood products (e.g., red cells, plasma, platelets). These substances are typically infused as (1) a single bolus volume (a few ml to several liters) over a limited time period (e.g., minutes to hours) or (2) a continuous infusion delivered a fixed or titrated rate (typical range 0.1 ml to 5 ml per minute).

Infusions can be administered through a variety of routes, most commonly intravenous but also intraarterial, subcutaneous, intrapleural, intraarticular, epidural, and intrathecal, intraperitoneal, and intramuscular. A wide variety of catheters are available to facilitate infusions in through these various routes. Although traditionally, infusions have been administered in hospital settings, an increasing number of patients are receiving infusions in ambulatory infusion centers and at home. Because these latter settings have fewer, less skilled clinical personnel, only certain infusions are deemed to be safe there such as intravenous antibiotics, certain chemotherapeutic agents, local anesthetics for postoperative pain control, and certain narcotic pain medications.

Healthcare infusions are generally driven by relatively stale technologies such as gravity, active displacement electric pumps, or non-electric pumps. All three have well known disadvantages. Gravity driven infusions have low capital and disposable costs but require careful monitoring by a nurse, are not very accurate, limit patient mobility, and have no patient safety features. Electric pumps are accurate (±3%), have built in safety features of debatable efficacy but are expensive, bulky, susceptible to human factors and limit mobility. Additionally, electronic infusion pump errors are a serious ongoing problem and represent a large share of the overall human and economic burden of medical errors. Electronic infusion pumps have become expensive and high maintenance devices, which have been plagued in recent years by recalls due to serious software and hardware problems. These pumps are designed for fine adjustments of infusions in complex patients, such as those in a critical care setting, and their use for routine infusions is technologic overkill. In terms of outpatient infusions, disposable pumps are convenient and fairly inexpensive but have no patient safety features and can be highly inaccurate (±15-40%) and are therefore unsuitable for use with medications where flow accuracy is critical, such as chemotherapeutic. The FDA's MAUDE database includes numerous reports of complications and even deaths resulting from disposable infusion pump flow inaccuracies.

The landmark 1999 Institute of Medicine report, “To Err is Human” (REF), attributed 40-100,000 deaths per year in the U.S. to medical errors. Medication errors, 40% of which are serious, life-threatening, or fatal, are the most common medical error and cost the health care system billions of dollars per year. Intravenous medication errors are the most common medication error and over 35% of these are related to infusion pumps. Studies have shown that despite progressively feature-laden “smart pumps”, human factors, software and hardware issue continue to contribute to serious errors (REF). The FDA's MAUDE Adverse Event reporting system contain numerous examples of serious injury and death related to infusion pump errors, both electric and disposable.

Thus, there is a need in the industry for improvements to simply non-electric pumps which can consistently provide safe and effective flow of fluid to a desired destination. Further, there is a need in the industry for improvements to managing flow rates of a fluid along a pathway. The present disclosure is directed toward solutions to address these needs, in addition to having other desirable characteristics.

### SUMMARY

In accordance with exemplary embodiments of the present disclosure, a system for delivering a constant flow rate of fluid along a pathway is provided. The system includes a pump having a central column around which an inflatable member is situated to accommodate a volume of fluid to be directed through the central column and along the pathway; a flow resistor being in fluid communication with the central column of the pump, the flow resistor including a piston movably situated within the flow resistor to define a constricted cross-sectional flow path, therebetween, for modifying a flow rate of a fluid flow through the flow resistor to passively regulate the fluid flow to a substantially constant flow rate; and the pathway being in fluid communication with an output of the flow resistor for directing the fluid flow to a site of interest at the substantially constant flow rate.

In accordance with exemplary aspects of the present disclosure, the pump can include at least one inner elastomer layer for containing the fluid at a predefined pressure and a protective outer sheath. The pump can include an outer elastomer layer disposed between the at least one inner elastomer layer and the protective outer sheath. In some examples, the pump cam include a central column having an input channel for directing fluid into the at least one inner elastomer layer to expand the at least one inner elastomer layer to generate the predefined pressure. The central column can include an output channel for providing the fluid along the pathway. The pump cam includes a plurality of coupling mechanisms to couple the at least one inner elastomer layer the central column in a fluid tight configuration. For example, the plurality of coupling mechanisms can be O-rings. The system can additionally include a plurality of end caps for coupling the protective outer sheath to the central column. The pump can include a central column having a recess and the flow resistor can be disposed within the recess in direct fluid communication with the pump. The pathway can include at least one filter disposed in-line configured to remove air from the fluid flowing through the pathway.

In accordance with exemplary embodiments of the present disclosure, an elastomeric pump is provided. The elastomeric pump can include an elastomer layer defining a cavity for receiving a volume of fluid; and a central column disposed at least partially within the cavity and in fluid communication with the cavity, a recess defined within the central column, the recess being sized and shaped for receiving a flow resistor, and the recess having an output channel for directing fluid out of the elastomeric pump to a destination site.

In accordance with some embodiments, the elastomeric pump can include an outer elastomer layer disposed between the elastomer layer and a protective outer sheath. The central column can extend proximally and distally through the elastomer layer. The elastomeric pump can additionally include plurality of coupling mechanisms to couple the elastomer layer to the central column in a fluid tight configuration. For example, the plurality of coupling mechanisms can be O-rings. The elastomeric pump can further include a plurality of end caps for coupling a protective outer sheath to the central column.

In some exemplary embodiments, the elastomeric pump can further include a flow resistor, the flow resistor can be disposed within the recess in direct fluid communication with the elastomeric pump. The flow resistor can include an inlet in fluid communication with the output channel to receive fluid from the cavity. The flow resistor can passively regulate the received fluid to output the fluid at a substantially constant flow rate, regardless of a flow rate of the received fluid. In some embodiments, the elastomeric pump further includes at least one seal disposed between the flow resistor and the recess. The flow resistor can be fully disposed within the recess.

In accordance with exemplary embodiments of the present disclosure, a method for delivering fluid to a site of interest is provided. The method includes selecting a flow resistor having a piston movably situated within the flow resistor to define a constricted cross-sectional flow path for modifying a flow rate through the flow resistor to a substantially constant flow rate; fluidly coupling the flow resistor downstream to a pump having a central column around which an inflatable member is situated to accommodate a volume of fluid; directing at least a portion of the volume of fluid through an output in the central column and along the constricted cross-sectional flow path of the flow resistor to the site of interest at the substantially constant flow rate.

In some embodiments, the inflatable member can apply a pressure to the volume of fluid to dispense the fluid through the central column. The pressure applied to the volume of fluid can be varied as the fluid is directed through the central column such that a flow rate of the fluid can increase as the volume of fluid in the inflatable member decreases. The method can further include introducing the fluid through the central column to inflate the inflatable member. Fluidly coupling the flow resistor can include inserting the flow resistor within a recess partially extending into the central column in direct fluid communication with the output in the central column.

### BRIEF DESCRIPTION OF THE FIGURES

These and other characteristics of the present disclosure will be more fully understood by reference to the following detailed description in conjunction with the attached drawings, in which:

FIG. 1 is front view of an example elastomeric pump assembly, in accordance with an embodiment of the present disclosure;

FIGS. 2A, 2B, and 2C are cross-sectional side views of flow resistors, in accordance with exemplary embodiments of the present disclosure;

FIG. 3 is a cross-sectional side view of an elastomeric pump system, in accordance with example embodiments of the present disclosure;

FIG. 4A is a front view of a partially assembled elastomeric pump, in accordance with example embodiments of the present disclosure;

FIG. 4B is a front view of a disassembled elastomeric pump, in accordance with example embodiments of the present disclosure, and

FIGS. 5A, 5B, 5C, and 5D are side view of a flow resistor being installed within a portion of an elastomeric pump, in accordance with example embodiments of the present disclosure.

### DETAILED DESCRIPTION

An illustrative embodiment of the present disclosure relates to a system for managing flow along a pathway. Specifically, the present disclosure relates to introducing a flow resistor within a system such that the fluid flow rate downstream from the flow resistor is substantially constant. The configuration of the present disclosure can be used in any combination of systems, for example, systems designed for pumping fluids. The flow resistor can be incorporated into the pump itself or downstream from the pump to ensure that the constant flow rate provided by the flow resistor is provided to the destination.

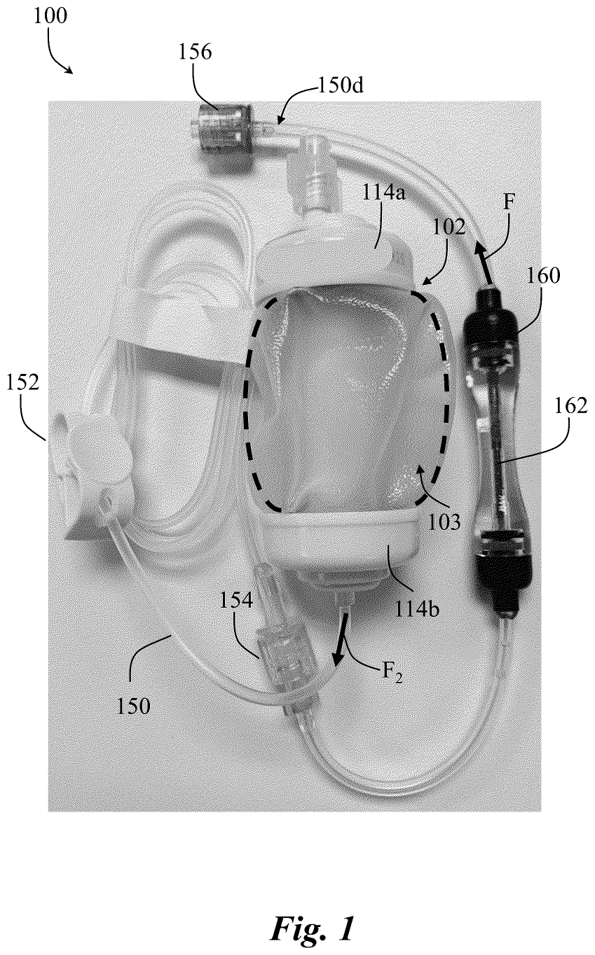

FIGS. 1 through 5D, wherein like parts are designated by like reference numerals throughout, illustrate an example embodiment or embodiments of improved system and/or operation for managing flow along a pathway, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

Referring to FIG. 1, in some embodiments, the present disclosure provides an assembly 100 for managing fluid flow along a pathway 150 to a site of interest. Generally, the assembly 100 can include an elastomeric pump 102 for dispensing a fluid and a flow resistor 160 to regulate and maintain a flow rate of the fluid at a fixed, or constant, flow rate. In some embodiments, the pathway 150 can include a tube, a catheter, or other structures having a lumen extending therethrough to allow a fluid to flow to a destination (not shown) or site of interest. The pathway 150, as will be described hereinafter, can include structures which can provide added functionality to the assembly 100. While the additional structures are shown in FIG. 1 in an exemplary arrangement, they can be disposed along the pathway 150 in any order as needed.

As illustrated in FIG. 1, in some embodiments, the pathway 150 can extend from the elastomeric pump 102. In some embodiments, the pathway 150 can include a clip 152 disposed on the pathway 150 to prevent or stop fluid from flowing through the assembly 100. For example, the clip 152 can be compressed to “pinch” the pathway 150 to create a substantially fluid tight seal preventing fluid from flowing through the pathway 150, without causing damage to the pathway 150. The clip 152 can include any combination of clips, clamps, or valves known in the art. In some cases, the clip 152 can be a valve disposed in-line with the pathway 150, or other structures to stop a flow of fluid.

Upstream, or downstream, of the clip 152, a filter 154 can be arranged in-line with the pathway 150 to remove unwanted components or particles within the fluid flowing through the pathway 150. In some examples, the filter 154 can be an air eliminating filter to ensure that air is not allowed to pass through the pathway 150 to the destination. The filter 154 can include any combination of filters known in the art.

At a distal end 150d of the pathway 150 a connector 156 can be disposed to allow the pathway 150 to be in fluid communication with a site of interest. For example, in an IV setup, the connector 156 can be a Luer fitting designed to mate with an intravenous needle disposed within a vein of a patient. Alternatively, the connector 156 can include any combination of connectors known in the art. Further, in some embodiments, it is contemplated that the assembly 100 can include additional components including valves, Y-connectors, injection port caps, flashback chambers, etc.

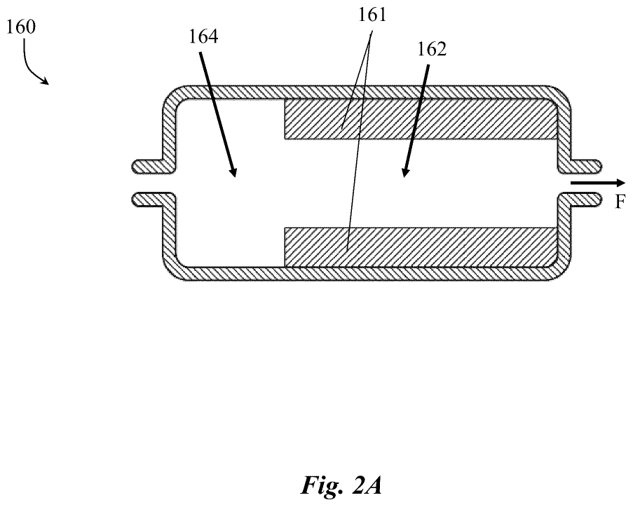

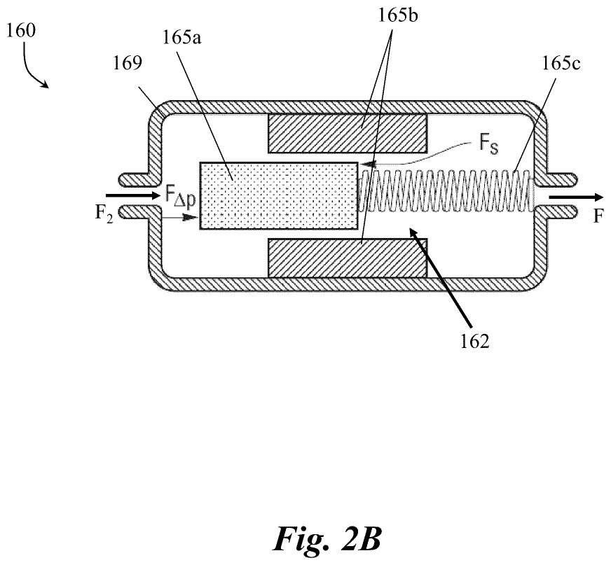

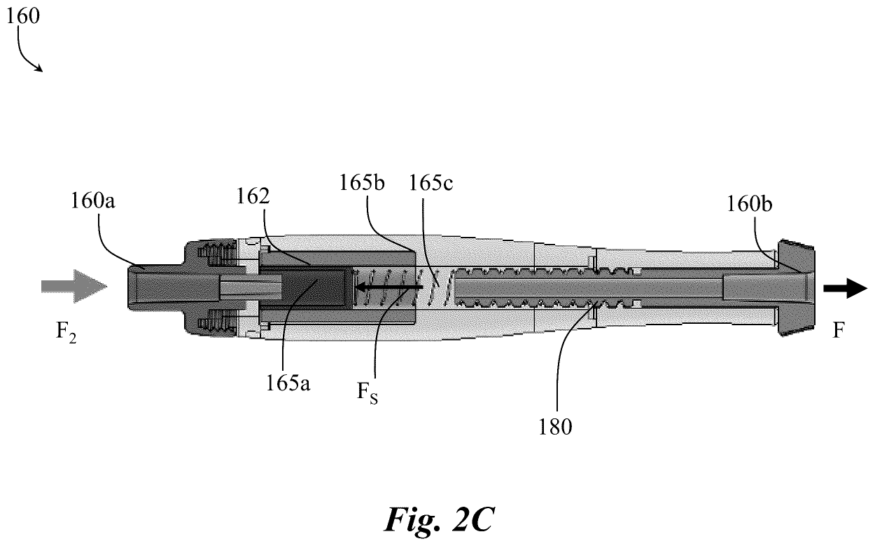

In some embodiments, as shown in FIG. 1, the assembly 100 can include a flow resistor 160 (or flow regulator) disposed in-line with the pathway 150. The flow resistor 160, in an embodiment, can be designed to maintain a flow of fluid through the pathway 150 at a fixed, or constant rate, regardless of an input flow rate from a fluid source, e.g., the elastomeric pump 102. As illustrated, the flow resistor 160 can be disposed at any location downstream of an output channel 122, as shown in FIG. 2, of the fluid source, e.g., fluidly coupled to the elastomeric pump 102. For example, as depicted in FIG. 1, the flow resistor 160 can be positioned in-line with the pathway 150 to receive a fluid flow from the elastomeric pump 102.

In some embodiments, the flow resistor 160 can be a flow resistor as discussed in U.S. application Ser. No. 16/845,752 or an adjustable flow resistor as discussed in PCT International Application PCT/US22/14834, both incorporated herein by reference in their entirety. For example, as shown in FIGS. 2A, 2B, and 2C, the flow resistor 160 can passively adjust a variable input flow rate to a constant, or fixed, output flow rate F. The flow resistor 160 can include a restricted flow path 162 to create laminar flow within the flow resistor 160 to passively slow an input flow from a fluid source, e.g., the elastomeric pump 102. In some cases, as shown in FIG. 2A, the restricted flow path 162 can be created only by static structures 161 disposed within the flow path 164 to form a restricted flow path 162 to create a reduced cross-sectional area, or constricted cross-sectional flow path, where the input flow under goes a laminar flow. In other embodiments, as shown in FIG. 2B, the restricted flow path 162 can be created by a movable element 165a, e.g., a piston, sliding within cylinder 165b of a fluid chamber 169, to create a variable reduced cross sectional area that defines a restricted flow path 162. The movable element 165a can move within the flow resistor 160 as a function of a pressure differential Fop between an input flow and a backflow pressure from the destination, e.g., a veinous pressure, as balanced by a force Fs of a resistive member 165c. With the movable element 165a and a resistive member 165c, the flow resistor 160 can self-adjust in situations where the input flow F₂ can have a rate that may vary.

In some embodiments, as shown in FIG. 2C, the flow resistor 160 can additionally include an adjustment mechanism 180 to set a biasing constant of the resistive member 165c to affect the constant, or fixed, output flow rate F. For example, the resistive member 165c can be provided with linear elastic properties (e.g., it obeys Hooke's Law such as conventional springs, elastomeric bands, etc.), to provide a custom and predefined relationship between an input pressure at the inlet 160a and an output pressure at the outlet 160b, such that the output flow F is one of a constant, or consistent, flow rate that is independent of any pressure differential between the inlet 160a and outlet 160b. The linear elastic properties can be defined by a biasing constant, e.g., a spring constant, for resistive members 165c which have elastic properties. As an example, the flow resistor 160 may have its inlet 160a be in fluid communication with a fluid source, e.g., the elastomeric pump 102, and its outlet 160b be in fluid communication with a vein of a patient. In such a setup, inlet 160a of the flow resistor 160 can be exposed to fluid pressure from input flow F₂ as it enters, while the outlet 160b of the flow resistor 160 can be exposed to a venous pressure from a patient's vein. These two pressures can define a pressure differential. The movable element 165a can further be exposed to a force Fs from resistive member 165c. The balance of the forces acting on the movable element 165a can determine the movement and location of the movable element 165a into and within the cylinder 165b to define the restricted flow path 162. By setting the biasing constant of the resistive member 165c with the adjustment mechanism 180, the force Fₛ can be changed to affect the length of the restricted flow path 162. With the length of the restricted flow path 162 changed, the resulting constant, or fixed, output flow rate F can be set to different values.

The flow resistor 160 can advantageously account for pressure, or flow rate, changes through the pathway 150. For example, when the flow resistor 160 is in fluid communication with the elastomeric pump 102 while it is infusing a fluid. In some examples, the elastomeric pump 102 relies upon pressure P created by an inner elastomer sleeve 106, or layer, and an outer elastomer sleeve 108, or layer. As fluid is released from the elastomeric pump 102, initially, the fluid flows at a consistent rate through the pathway 150. However, as the elastomeric pump 102 is emptied of liquid, the pressure of the fluid flow can increase, or spike, which can result in a higher fluid flow rate. Thus, in order to ensure that the fluid flow F is consistent, the flow resistor 160 can be disposed downstream of the elastomeric pump 102 to ensure that the flow rate of the fluid flow remains consistent.

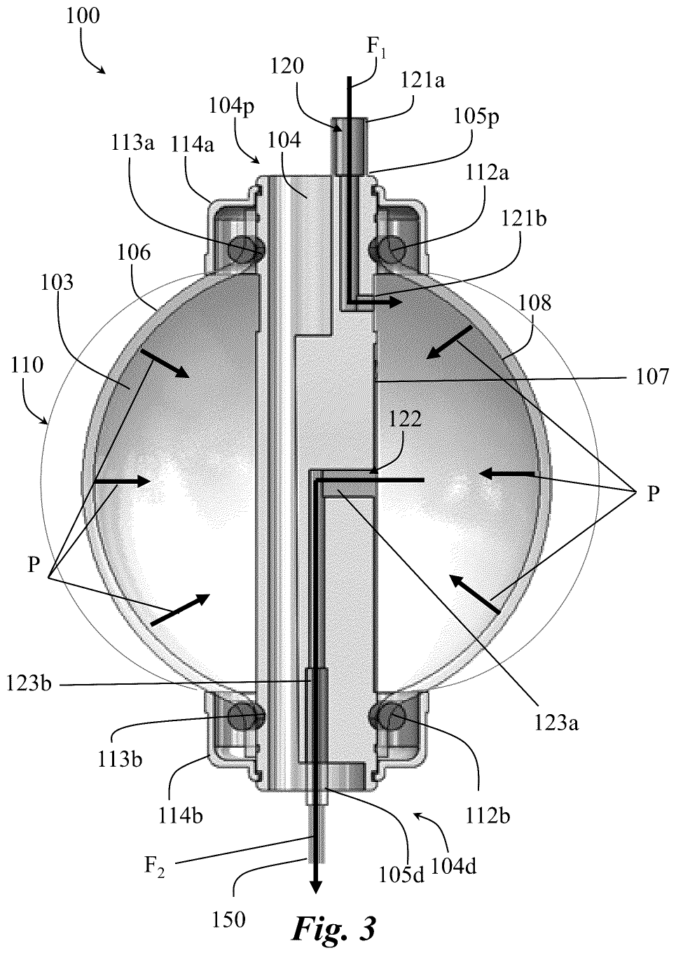

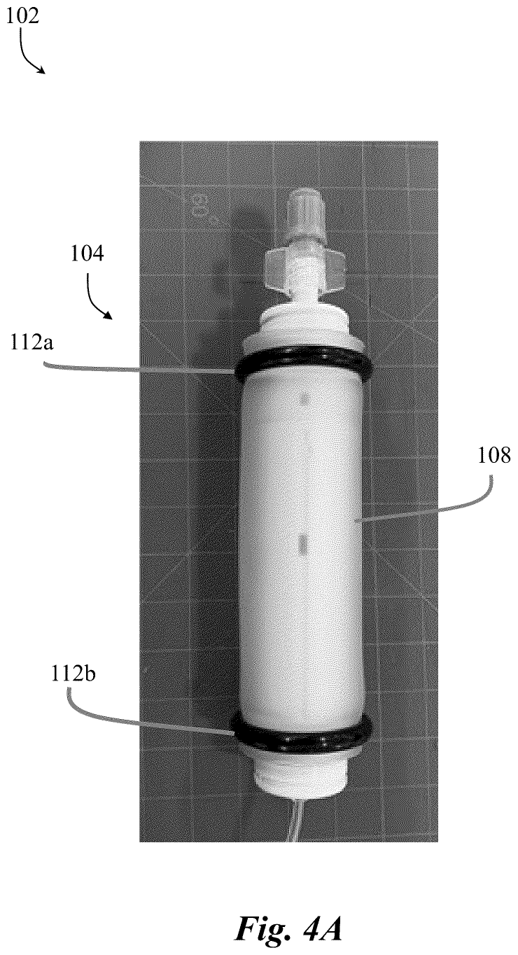

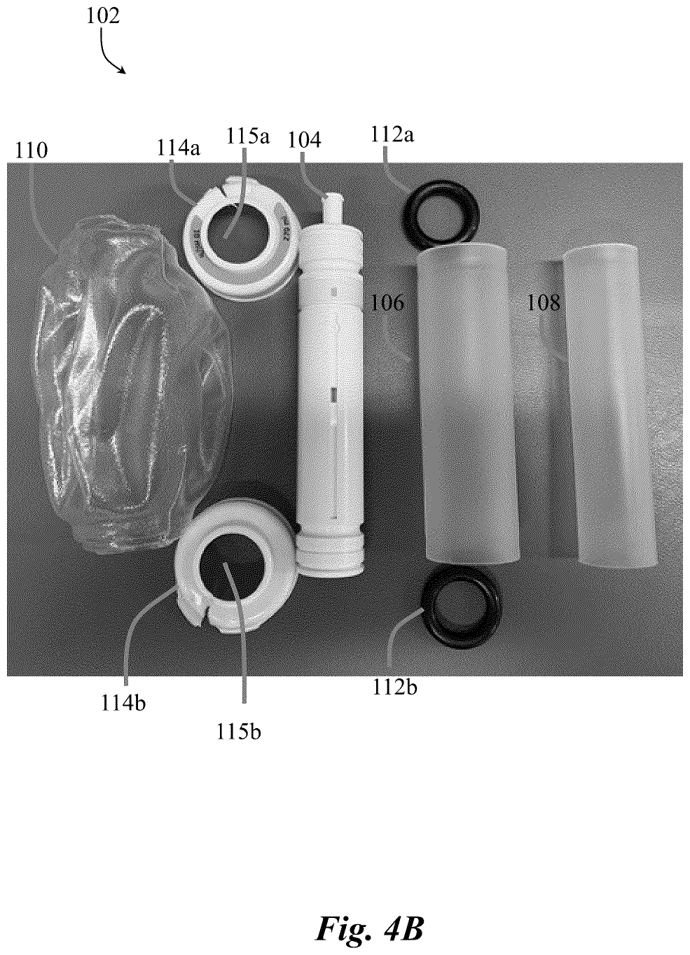

Referring to FIGS. 3, 4A, and 4B, the elastomeric pump 102 according to an exemplary embodiment is shown. The elastomeric pump 102 can include a central column 104, or central support, which can retain an inner elastomer sleeve 106 and an outer elastomer sleeve 108 to define a cavity 103. The inner and outer elastomer sleeves 106, 108 can be inflated with a fluid filling the cavity 103. As the cavity 103 is filled a volume of fluid, the inner and outer elastomer sleeves 106, 108 can apply a pressure to allow the fluid to be passively pumped through the pathway 150.

The inner and outer elastomer sleeves 106, 108 can be generally cylindrical in shape when not inflated. Alternatively, the inner and outer elastomer sleeves 106, 108 can have other non-inflated shapes including a spherical shape. In some embodiments, the inner and outer elastomer sleeves 106, 108 can have substantially the same size and shape. Alternatively, the inner and outer elastomer sleeves 106, 108 can have different sizes and shapes. In the illustrated embodiment, the outer elastomer sleeve 108 can provide extra protection from leaks. While two elastomer sleeves, the inner and outer elastomer sleeves 106, 108, are illustrated, the elastomeric pump 102 can be provided with any number of elastomer sleeves. The elastomeric sleeves, in an embodiment, can be made from any compliant material such that the inner and outer elastomer sleeves 106, 108 can be stretched to create a pressure P when filled with a sufficient quantity of fluids. It is contemplated that the inner and outer elastomer sleeves 106, 108 may be made from the same material having the same material properties; or, alternatively, the inner and outer elastomer sleeves 106, 108 can be formed of different materials to permit for specific performance of the elastomeric pump 102. Each of the inner and outer elastomer sleeves 106, 108 can be sufficiently compliant to expand to a desired size and shape to provide a desired flow rate. The thickness, diameter, elastomeric qualities, etc. of the inner and outer elastomer sleeves 106, 108 can be selected to provide a pre-selected flow rate.

The inner elastomer sleeve 106, in one embodiment, can be disposed about the central column 104 and extend from about a proximal end 104p to about a distal end 104d. The outer elastomer sleeve 108 can similarly be disposed about the inner elastomer sleeve 106 and extend from about the proximal end 104p to about the distal end 104d of the central column 104. In some embodiments, the inner and outer elastomer sleeves 106, 108 can be sealed in substantially fluid tight engagement to the central column 104 at the proximal end 104p and the distal end 104d. The fluid tight seal can be accomplished via any mechanical or chemical means. For example, as shown in FIGS. 3 and 4A, the inner and outer elastomer sleeves 106, 108 can be sealed to the column with a first, proximal, O-ring 112a and a second, distal, O-ring 112b. The 112a, 112b can be advanced over each of the ends of the central column 104 and the inner and outer elastomer sleeves 106, 108 until they reach and compress into respective recesses 113a, 113b formed about the central column 104.

In some embodiments, the elastomeric pump 102 can include a protective sheath 110, or protective outer sheath, surrounding both the inner and outer elastomer sleeves 106, 108. The protective sheath 110 can be constructed from a different material than the inner and outer elastomer sleeves 106, 108. For example, the protective sheath 110 can be constructed from a non-compliant plastic material that is sufficiently sized to allow the inner and outer elastomer sleeves 106, 108 to expand to a desired inflation, as shown in FIG. 3. In some embodiments the protective sheath 110 can be sized and shaped to allow the inner and outer elastomer sleeves 106, 108 to expand without restriction.

The protective sheath 110 can be secured to the central column 104 to create a protective barrier in the case that a fluid leaks from the inner and outer elastomer sleeves 106, 108. As illustrated in FIG. 3, a proximal end cap 114a can be coupled to the proximal end 104p of the central column 104 to secure the protective sheath 110 in place about the proximal end 104p. Similarly, a distal end cap 114b can be coupled to the distal end 104d of the central column 104 to secure the protective sheath 110 to the distal end 104d. In some examples, the end caps 114a, 114b can be screwed or friction fit over the proximal end 104p and distal end 104d of the central column 104. As illustrated in FIG. 4B, the end caps 114a, 114b can each include a central opening 115a, 115b to receive the proximal end 104p and the distal end 104d of the central column.

The combination of the O-rings 112a, 112b and the end caps 114a, 114b can apply sufficient force against the central column 104 to maintain a fluid tight seal between the central column 104 and the elastomer sleeves 106, 108 as fluid is added to the cavity 103 and pressure P increases. As would be appreciated by one skilled in the art, any combination of coupling mechanisms can be used in addition to or in place of the O-rings 112a, 112b and the end caps 114a, 114b.

The central column 104 can be a generally cylindrical structure having sufficient rigidity to maintain structural integrity when the elastomeric pump 102 is filled with a fluid. At a proximal end 104p of the central column 104 an input channel 120 can be provided to introduce a fluid into the elastomeric pump 102. As illustrated, the input channel 120 can have a generally “L” shaped channel extending from an inlet 121a to an outlet 121b, however other shaped input channels 120 are considered to be within the scope of this disclosure. The inlet 121a can extend through a proximal face 105p of the central column 104 and the outlet 121b can be disposed distal to O-ring 112a and can extend through an outer surface 107 of the central column 104. The input channel 120 can provide a fluid pathway to allow an input flow F₁ to enter into a cavity 103 created between the inner elastomer sleeve 106 and the central column 104.

At a distal end 104d of the central column 104, an output channel 122 can be provided to allow the fluid to exit the elastomeric pump 102. In some embodiments, the output channel 122 can have a generally “L” shaped channel extending from an inlet 123a to an outlet 123b, however other shaped output channels 122 are considered to be within the scope of this disclosure. The outlet 123b can be disposed through a distal face 105d of the central column 104. The inlet 123a can be disposed proximal to the distal O-ring 112b and can extend through the outer surface 107 of the central column 104. The outlet 123b can receive, or be in fluid communication, with the pathway 150 of the assembly 100. The output channel 122 can provide a fluid pathway to allow an output flow F₂ to exit the elastomeric pump 102 into the pathway 150. While not shown, the output channel 122 can additionally include a valve, or bung, to prevent the output flow F₂ from exiting the elastomeric pump 102 while the elastomeric pump 102 is being filled. Alternatively, if the elastomeric pump 102 is in fluid communication with the pathway 150 before being filled, the clip 152 can be activated to prevent fluid from exiting the assembly 100 while the elastomeric pump 102 is being filled.

In use, the elastomeric pump 102 can operate by being filled with fluid, such as medication, to expand the inner and outer elastomer sleeves 106, 108. As the inner and outer elastomer sleeves 106, 108 are being expanded, and stretched, the elastic properties of the inner and outer elastomer sleeves can generate a pressure P against the fluid. Once the elastomeric pump 102 has been filled with a fluid, the input channel 120 can be sealed to prevent fluid from exiting the elastomeric pump 102 in a flow opposite to the input flow F₁. Then, the pressure P of the inner and outer elastomer sleeves 106,108 can force the fluid through the output channel 122 to the pathway 150 once the fluid is allowed to flow. For example, clip 152 can be released from the pathway 150 to allow the output flow F₂ to exit the outlet 123b of the elastomeric pump 102.

As the output flow F₂ continues to exit the elastomeric pump 102, the pressure P will eventually increase, thus increasing the flow rate of the output flow F₂ through the pathway 150. In some situations, it may be undesirable to have an output flow F₂ that has a variable flow rate. In that case, a flow resistor 160 can be provided in-line with the pathway 150. As discussed above, the flow resistor 160 can advantageously provide a passive mechanism to adjust a flow rate of the output flow F₂ to maintain a constant fixed flow rate F through the pathway 150. Thus, the combination of the elastomeric pump 102 and the flow resistor 160 can provide for an inexpensive and reliable assembly to deliver an output flow F₂ with a constant fixed flow rate.

The elastomeric pump 102 can also be used along with any combination of mechanisms that can generate a pressure. For example, the elastomeric pump 102 can include a spring and a piston to generate the driving pressure or be provided with a gas cartridge to generate a pressure on the fluid being infused. In one embodiment, the elastomeric pump 102 can be designed to be portable and simple to setup without requiring additional mechanical and electrical hookups and/or programming to operate.

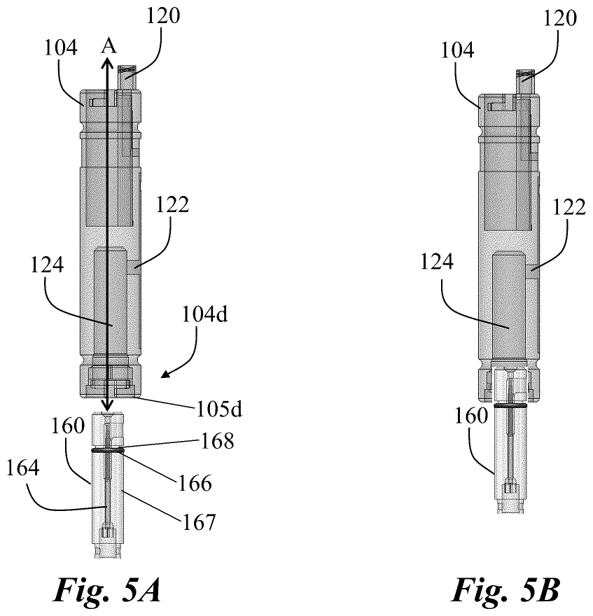

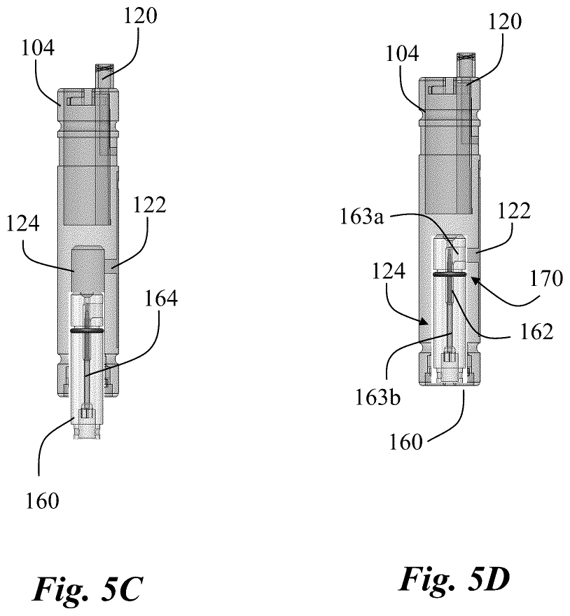

FIGS. 5A-5D show an alternative assembly 100. The assembly 100 of FIGS. 5A-5D can be substantially the same as the assembly of FIGS. 1, 3, 4A, and 4B. However, instead of a flow resistor 160 being downstream of the elastomeric pump 102 in-line with a pathway 150, the elastomeric pump 102 of FIGS. 5A-5D can be designed to receive and/or directly couple with the flow resistor 160. Thus, only the structure pertaining to the coupling of the flow resistor 160 and the elastomeric pump 102 will be discussed. All the other structure of the assembly 100 including the elastomeric pump 102 and the flow resistor 160 is the same and will not be discussed for the sake of brevity.

In some embodiments, the present disclosure can provide for a condensed assembly 100 in which the elastomeric pump 102 includes a recess 124 extending within a central column 104. As noted above, the central column 104 can provide the base structural element for the elastomeric pump 102. In contrast to the “L” shaped output channel 122 of the embodiment of FIGS. 1, 3, 4A, and 4B, the output channel 122 of FIGS. 5A-5D can be a straight channel that extends through the outer surface 107 towards a central axis A of the central column.

The central column 104 can further include a recess 124. The recess 124 can extend proximally through a distal face 105d to intersect with, and be in fluid communication with, the output channel 122. In some embodiments, the recess 124 can be sized and shaped to receive the flow resistor 160 to form a fluid tight seal 170. For example, as illustrated in FIGS. 5A-5D, the flow resistor 160 can be inserted into the distal end 104d of the central column 104. Alternatively, the flow resistor 160 can be axially aligned, and in direct contact, with the central column 104, end-to-end, to allow fluid to be directed from within a cavity created by the two elastomer sleeves (not shown in FIGS. 5A-5D).

The flow resistor 160 can, in some embodiments, have a substantially “L” shaped flow path 164. The flow path 164 can include an input path 163a and an output path 163b. The input path 163a can extend perpendicular to a central axis of the flow resistor 160. When the flow resistor 160 is fully inserted into the recess 124, as seen in FIG. 5D, the input path 163a can be in direct, or indirect, fluid communication with the output channel 122 of the central column 104. The input path 163a can extend from the output channel 122 towards the central axis of the flow resistor 160 and then take a perpendicular turn to the output path 163b. In some embodiments, the output path 163b can include structure, for example a restricted flow path 162, to passively adjust the flow rate of a fluid flowing through the flow resistor 160, as discussed above, such that a change in fluid pressure or flow rate from the elastomeric pump 102 does not result in a change of flow rate of the fluid at the destination. Alternatively, the restricted flow path 162 can be arranged anywhere along the flow path 164.

As shown in FIGS. 5A-5D, the flow resistor 160 can include a sealing element 166 to create a seal 170 between the flow resistor 160 and the central column 104. The sealing element 166 can be disposed between the flow resistor 160 and the central column 104 at a location distal to the output channel 122. In some embodiments, the sealing element 166 can be a gasket or O-ring, disposed about an outer surface 167 thereof. In some embodiments, as shown in FIG. 5A, the outer surface 167 can include a groove 168 to retain the sealing element 166. The sealing element 166 can have an outer dimension, or circumference, which is larger than the inner dimension of the recess 124 such that a fluid tight seal 170 is created, as seen in FIG. 5D. For example, the sealing element 166 can be compressive to create an interference fit as the sealing element “pushes” against the recess 124, as the flow resistor 160 is inserted into the recess 124 as progressively shown in FIGS. 5A-5D. Alternatively, the recess 124 can include the sealing element 166 on the inner surface thereof, to create a fluid tight seal between the flow resistor 160 and the output channel 122 of the central column 104.

In some embodiments, the flow resistor 160 can be removably coupled to the central column 104 within the recess 124. The flow resistor 160 and the recess 124 can share complimentary structures, not shown, which can be designed to mate with one another to form a mechanical coupling. For example, the flow resistor 160 can include pins that mate with a channel within the recess 124 such that twisting or rotating the flow resistor 160 within the recess 124 can create a mechanical bond with the central column 104. Further, the flow resistor 160 can also be decoupled and removed from the recess 124 in a similar manner, for example, by rotating the flow resistor 160 in an opposing direction. As would be appreciated by one skilled in the art, any combination of mechanical or chemical couplings can be provided to couple the flow resistor 160 to the central column 104. For example, the coupling can be a friction fit, screw in fit, snap in fit, adhesive, etc.

Once the flow resistor 160 has been fully seated within the recess 124 of the central column 104, the input flow path 163a can be in fluid communication with the output channel 122 of the central column 104. In the fully seated configuration, the flow resistor 160 can be fully received within the central column 104 to create an integrated device 200 that combines the flow resistor 160 and the elastomeric pump 102. Advantageously, the elastomeric pump 102 can be packaged with a variety of different flow resistors 160 to permit a user to pre-select a desired fixed output flow from the integrated device 200.

In use, a user can determine what fixed output flow rate is needed for a given destination. Upon making that determination, the user can select an appropriately sized flow resistor 160 and insert the flow resistor 160 within a recess 124 of an elastomeric pump. Alternatively, the elastomeric pump 102 can be pre-packaged with a single flow resistor 160 which can be pre-chosen for a given application. The user can then couple a pathway 150, e.g., medical grade tubing, to an output path 163b of the flow resistor 160 and close off the pathway 150 to prevent fluid from passing therethrough. Once the pathway 150 is closed off, a fluid can be introduced into the elastomeric pump 102 through the input channel 120 to expand the elastomeric pump 102, and the input channel 120 can be sealed. For example, as fluid is introduced into the elastomeric pump 102 it will fill a cavity 103 created by inner and outer elastomer sleeves 106, 108 until an expansion threshold is achieved. In other words, the inner and outer elastomer sleeves 106, 108 can be in a deflated state, as shown in FIG. 1, before the introduction of a fluid and can transition to an inflated state, as shown in FIG. 3, after the fluid is introduced into the cavity 103. Thereafter, the inner and outer elastomer sleeves 106, 108 can apply pressure to direct the fluid toward an input of the flow resistor 160. The pathway 150 can then be opened to permit a fluid to flow out the output channel 122 and into the flow resistor 160 such that the flow rate of the fluid is regulated to a fixed output flow through the pathway 150. In some embodiments, the pathway 150 can be closed, or opened, with a clip 152 disposed downstream of the elastomeric pump 102.

As fluid continues through the pathway 150, the flow resistor 160 can outputs the fluid flow at a substantially constant, fixed, flow rate, regardless of the received rate of flow from the elastomeric pump. Therefore, regardless of the flow rate output by the elastomeric pump 102 and/or variations in the flow rate provided by the pump 102, the flow resistor 160 can alter the flow rate such that there is a substantially constant, fixed, flow rate being delivered to a destination. For example, the elastomeric pump 102 can provide a fluid flow at a first flow rate initially. As the elastomeric pump 102 is emptied, the pressure may increase thereby increasing the flow rate of the fluid to a second, faster, flow rate. Thus, the flow resistor 160 can adjust the second, faster, flow rate to the desired flow substantially constant, fixed, flow rate for the particular application (e.g., delivering fluids to a patient).

The assembly 100 can be designed and/or modified to be used for various applications. For example, the assembly 100 can be used for delivering fluids to a patient, such as intravenous (IV) infusions of saline, medications, antibiotics, or other drugs. Alternatively, the assembly 100 can be implemented as part of a water pump system to regulate a flow of water being delivered, regardless of changes in fluid flow being applied by the water pump.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A system for delivering a constant flow rate of fluid along a pathway, the system comprising:
a pump having a central column around which an inflatable member is situated to accommodate a volume of fluid to be directed through the central column and along the pathway;
a flow resistor being in fluid communication with the central column of the pump, the flow resistor including a resistive member and a moveable element situated within the flow resistor, the moveable element configured to move within the flow resistor and to define a constricted cross-sectional flow path between an inner surface of the flow resistor and the moveable element, the resistive member having an elastic property and configured to affect the movement of the moveable element, the flow resistor configured to modify a flow rate of a fluid flow through the flow resistor to passively regulate the fluid flow to a substantially constant flow rate; and
the pathway being in fluid communication with an output of the flow resistor for directing the fluid flow to a site of interest at the substantially constant flow rate.

2. The system of claim 1, wherein the pump includes at least one inner elastomer layer for containing the fluid at a predefined pressure and a protective outer sheath.

3. The system of claim 2, wherein the pump includes an outer elastomer layer disposed between the at least one inner elastomer layer and the protective outer sheath.

4. The system of claim 2, wherein the central column includes an input channel for directing fluid into the at least one inner elastomer layer to expand the at least one inner elastomer layer to generate the predefined pressure.

5. The system of claim 4, wherein the central column includes an output channel for providing the fluid along the pathway.

6. (canceled)

7. (canceled)

8. (canceled)

9. The system of claim 1,
wherein the central column includes a recess, the recess sized and shaped for receiving the flow resistor, the recess having an output channel for directing fluid out of the elastomeric pump to a destination site; and
the flow resistor configured to removably couple to the central column within the recess in direct fluid communication with the pump.

10. The system of claim 1, wherein the pathway includes at least one filter disposed in-line configured to remove air from the fluid flowing through the pathway.

11. An elastomeric pump comprising:
an elastomer layer defining a cavity for receiving a volume of fluid;
an outer elastomer layer disposed between the elastomer layer and a protective outer sheath; and
a central column disposed at least partially within the cavity and in fluid communication with the cavity, a recess defined within the central column, the recess being sized and shaped for receiving a flow resistor, and the recess having an output channel for directing fluid out of the elastomeric pump to a destination site.

12. (canceled)

13. The elastomeric pump of claim 11, wherein the central column extends proximally and distally through the elastomer layer.

14. (canceled)

15. (canceled)

16. (canceled)

17. The elastomeric pump of claim 11 further including the flow resistor being disposed within the recess in direct fluid communication with the elastomeric pump.

18. The elastomeric pump of claim 17,
wherein the flow resistor includes an inlet in fluid communication with the output channel to receive fluid from the cavity, and
wherein the flow resistor passively regulates the received fluid to output the fluid at a substantially constant flow rate, regardless of a flow rate of the received fluid.

19. The elastomeric pump of claim 17, further comprising at least one seal disposed between the flow resistor and the recess.

20. The elastomeric pump of claim 17, wherein the flow resistor is fully disposed within the recess.

21. A method for delivering fluid to a site of interest, the method comprising:
selecting a flow resistor having a resistive member and a moveable element situated within the flow resistor, the moveable element configured to define a constricted cross-sectional flow path, the resistive member having an elastic property and configured to affect the movement of the moveable element, the flow resistor configured to modify a flow rate through the flow resistor to a substantially constant flow rate;
fluidly coupling the flow resistor downstream to a pump having a central column around which an inflatable member is situated to accommodate a volume of fluid;
directing at least a portion of the volume of fluid through an output in the central column and along the constricted cross-sectional flow path of the flow resistor to the site of interest at the substantially constant flow rate.

22. The method of claim 21, wherein the inflatable member applies a pressure to the volume of fluid to dispense the fluid through the central column.

23. The method of claim 22, wherein the pressure applied to the volume of fluid is varied as the fluid is directed through the central column such that a flow rate of the fluid increases as the volume of fluid in the inflatable member decreases.

24. The method of claim 21, further comprising,
introducing the fluid through the central column to inflate the inflatable member.

25. The method of claim 21, wherein the fluidly coupling the flow resistor step further includes inserting the flow resistor within a recess partially extending into the central column in direct fluid communication with the output in the central column.

26. The method of claim 21, wherein:
the flow resistor further comprises an adjustment mechanism configured to adjust the elastic property of the resistive member and controllably modify the substantially constant output flow rate;
the method further comprising, adjusting the elastic property of the resistive member to set the substantially constant output flow rate to a different substantially constant rate.

27. The system of claim 1, wherein the flow resistor further comprises an adjustment mechanism configured to adjust the elastic property of the resistive member, and controllably modify the substantially constant output flow rate to a different substantially constant rate.
